(19) United States
(12) Patent Application Publication
(10) Pub. No.: US 2007/0053869 A1
(43) Pub. Date: Mar. 8, 2007

(54) Formulation and method for enhancement of gastrointestinal absorption of pharmaceutical agents

(76) Inventors: Yuichi Sugiyama, Chiba (JP); Mariko Morishia, Kawasaki (JP); Hassan Benameur, Colmar (FR); Roland Daumesnil, Callian (FR)

Correspondence Address: Rosemary M. Miano;Warner-Lambert Company LLC, 201 Tabor Road, Morris Plains, NJ, 07950, US

(21) Appl. No.: 11/509,355

(22) Filed: Aug. 24, 2006

Related U.S. Application Data
(60) Provisional application No. 60/713,343, filed on Sep. 2, 2005.

(51) Int. Cl. A61K 31/765; A61K 31/4745; A61K 31/336

(52) U.S. Cl. 424/78.38; 514/283; 514/475

(57) ABSTRACT

The present invention relates to a method of enhancing absorption of a pharmaceutical agent by administering the agent in combination with an inhibitor of BCRP/ABCG2 wherein the amount of the inhibitor is about the critical micelle concentration of the inhibitor or less than the critical micelle concentration. The invention also relates to a formulation suitable for use to enhance absorption of a pharmaceutical agent. The pharmaceutical agent can be a chemotherapeutic agent. The invention also relates to capsules containing the formulation.

### CROSS-REFERENCE TO RELATED APPLICATION

This application claims the benefit of U.S. Provisional Patent Application No. 60/713,343, filed Sep. 2, 2005, which is hereby incorporated herein.

### FIELD OF THE INVENTION

The present invention provides a formulation and method for enhancing gastro-intestinal absorption of a pharmaceutical agent by inhibiting the active efflux transporter BCRP/ABCG2. Moreover, the invention provides pharmacologically active excipients and methods of using them for the inhibition of BCRP/ABCG2. The invention further provides pharmaceutical agents, such as chemotherapeutic agents suitable for use with the excipients of the invention.

### BACKGROUND OF THE INVENTION

The ATP-binding cassette (ABC) proteins are a large protein family of about 48 members. The “full transporters” have four domains on one polypeptide chain: two transmembrane domains and two nucleotide-binding domains. Each transmembrane domain spans the plasma membrane six times. The “half transporters” have two domains: a transmembrane domain and a nucleotide domain. “Half transporters” become active after dimerization. The ABC proteins use the energy released by hydrolysis of ATP by the ABC nucleotide domain to transport their substrate(s) against a concentration gradient.

The breast cancer resistance protein (BCRP, systematically known as ABCG2) belongs to the ABC family of drug half-transporters. Recently, ABCG2 has been shown to be expressed in many normal tissues, for instance, at the apical membrane of placental syncytiotrophoblasts, at the bile canalicular membrane of hepatocytes, and at the luminal membranes of villous epithelial cells in the small intestine and colon. The localization of ABCG2 suggests that it could have a potential role in protecting the tissues against the exposure to xenobiotics by extruding them across the apical membrane.

Recently, it has been reported that several pharmaceutical excipients can inhibit the function of P-glycoprotein (P-gp) in the intestine, therefore increasing the oral absorption of P-gp substrates. Johnson et al. reported inhibitory effects of polyethylene glycol 400, Pluronic P85, and D-α-tocopheryl polyethylene glycol 1000 succinate (TPGS) on the P-glycoprotein (P-gp/ABCB1). Johnson, Charman, and Porter, An In Vitro Examination of the Impact of Polyethylene Glycol 400, Pluronic P85, and Vitamin E d-α-Tocopheryl Polyethylene Glycol 1000 Succinate on P-Glycoprotein Efflux and Enterocyte-Based Metabolism in Excised Rat Intestine, AAPS PharmSci 2002:4, 1. P-gp is a full transporter. Cornaire et al. reported enhancement of absorption of digoxin by several excipients, including Labrasol, Imwitor 742, Acconon E, Softigen 767, Cremophor EL, Miglyol, Solutol HS 15, sucrose monolaurate, polysorbate 20, TPGS, and polysorbate 80. Cornaire, Woodley, Hermann, Cloarec, Arellano, and Houin, Impact of excipients on the absorption of P-glycoprotein substrates in vitro and in vivo, Int. J. Pharm. 2004, 278, 119.

In normal tissue, high expression of the ABCG2 is found in the epithelial cells of both small and large intestines. The localization of ABCG2 suggests that it could have a potential role in protecting the tissues against the exposure to xenobiotics by extruding them across the apical membrane. Drugs that would be substrates of ABCG2 have low absorption in the digestive tract and this can lead to low bioavailability of the drug.

This invention addresses the issue of drug dosing and availability by evaluating the role of certain pharmaceutical excipients in the inhibition of ABCG2 function. Inhibition of ABCG2 function could possibly improve the absorption of ABCG2 substrate drugs from the digestive tract. Therefore, we examined whether some of the currently used pharmaceutical excipients inhibit ABCG2 function.

### SUMMARY OF THE INVENTION

The invention relates to formulations and methods for increasing the uptake of pharmaceutically active agents by inhibiting the ABCG2 transport system. The formulations of the invention are suitable for enteric use and use with other mucosal surfaces. In one aspect, the invention provides a benefit in the effective formulation and use of drugs that are subject to efflux by the ABCG2 transport system by identifying useful inhibitors of ABCG2 and methods of their use.

One aspect of the invention is a method of enhancing absorption of a pharmaceutical agent comprising administering said agent to a subject in need of such treatment, in combination with an inhibitor of ABCG2 particularly wherein the amount of the excipient can be at a value less than or at the critical micelle concentration (cmc) of the inhibitor when delivered enterically. In one particular aspect, the amount of the excipient can be at a value below the cmc. In another particular aspect, the amount of the excipient can be at a value above the cmc. In still another aspect, the amount of the excipient is at a value at or above the cmc. The agent can be administered to a gastrointestinal tract of the subject. The excipient can be selected from a wide range of ABCG2 inhibitors, including, but not limited to Macrogol esters (Polyoxyl 35 Castor oil), Macrogol sorbitan esters (Polysorbate 20), Macrogol alkyl ethers (Polyoxyl 4 lauryl ether), Ethylene Oxide/Propylene Oxide Block Copolymer; (PEO)26(PPO)39.5(PEO)26, Pluronic L81, Macrogol sorbitan esters (polyoxyethylenesorbitan monooleate), lauryl maltopyranoside (LM), Macrogol esters (Polyoxyl 40 stearate), Macrogol esters (Polyoxyl 40 hydrogenated castor oil), Vitamin E TPGS, Poloxamer 188, and mixtures thereof and can include combinations of ABCG2 inhibitors. A generic description of the foregoing excipients is found in Table 1. The pharmaceutical agent can be any pharmaceutical agent, including, but not limited to, a chemotherapeutic agent.

Another aspect of the invention is a method of enhancing absorption of a pharmaceutical agent comprising administering said agent in combination with reserpine, CI 1033, GF 120918, fumitremorgin C (FTC), Ko 134 or Ko 132 and an excipient, particularly wherein the resulting concentration of said excipient is less than or at the critical micelle concentration.

The inhibitors of the invention are useful for enhancing absorption of drugs that are subject to efflux by ABCG2. In a particular aspect, the beneficial excipients of the invention overcome the action of ABCG2. The excipients may be substrates of ABCG2, but the invention does not rest on a particular molecular mechanism. In one aspect, the method is directed to enhancement of absorption of a pharmaceutical agent when P-gp/ABCB1 is substantially inhibited.

The invention is also directed to a method of enhancing absorption of a pharmaceutical agent.

Yet another aspect of the invention is a method of enhancing absorption of a pharmaceutical agent comprising administering said agent in combination with an amount of an excipient which results in inhibition of ABCG2 function. In one particular aspect there is at least about 30%, more particularly about 40%, and even more particularly about 60 % inhibition of ABCG2.

In one aspect, the invention comprises a composition for mucosal administration comprising a pharmaceutical agent and an excipient capable of inhibiting ABCG2 particularly wherein the concentration of said excipient resulting from the administration is below or substantially below the critical micelle concentration of said excipient. In yet another aspect the concentration of the excipient upon administration is at or below the cmc. In another aspect, the concentration of the excipient upon administration is at or above the cmc. In yet another aspect, the concentration of the excipient upon administration is substantially at the cmc. The composition can be an oral dosage form. In a further aspect, the oral dosage form can have a concentration upon administration of said excipient of about one-half the critical micelle concentration of said excipient. The oral dosage form can also have a concentration upon administration of said excipient of about one-quarter the critical micelle concentration of said excipient. The oral dosage form can also have a concentration upon administration of said excipient of about one-eighth the critical micelle concentration of said excipient. In one embodiment the oral dosage form has a concentration upon administration of said excipient between about one-eighth of the cmc and about the cmc. In another embodiment the oral dosage form has a concentration upon administration of said excipient between about one-eighth and one-half of the cmc. In a particular aspect, the amount of excipient upon administration is between about one-eighth and about one-quarter of the cmc. In another particular aspect, the amount of excipient upon administration is between about one-quarter and about one-half of the cmc. In another particular aspect, the amount of excipient upon administration is between about one-half of the cmc and the cmc.

In another aspect, the invention comprises a pharmaceutical formulation for the treatment of a subject in need thereof comprising an effective amount of a pharmaceutical agent and an excipient, particularly wherein the excipient is present in an amount that is substantially below the critical micelle concentration when delivered enterically. In yet another aspect of the invention, the excipient is present in an amount that is above the cmc. In still another aspect of the invention, the excipient is present in an amount that is at or above the cmc. In yet still another aspect of the invention, the excipient is present in an amount that is below the cmc. In even another aspect of the invention, the excipient is present in an amount that is at or below the cmc. The invention can further comprise a capsule comprising the formulation. The agent of the formulation can be a chemotherapeutic agent.

The invention can also comprise a capsule comprising a pharmaceutical agent and an excipient wherein the concentration of said excipient is at the critical micelle concentration of said excipient.

In one aspect, the invention comprises a method of enhancing absorption of a pharmaceutical agent comprising administering said agent to a subject in combination with an inhibitor of ABCG2, wherein the amount of the inhibitor is less than or about the critical micelle concentration of the inhibitor upon dilution into 200 ml of fluid. The critical micelle concentration can be measured by surface tension. The fluid can be selected from the group consisting of water, buffer, natural or simulated stomach fluid, and natural or simulated intestinal fluid. In one particular aspect, the fluid is water. In one aspect, the inhibitor can be selected from the group consisting of polyoxyethyleneglyceroltriricinoleate 35; polyoxyethylenesorbitan monolaurate; lauryl polyethylene glycol ether; ethylene oxide/propylene oxide block copolymer (PEO)₂₆(PPO)_{39.5}(PEO)₂₆; and ethylene oxide/propylene oxide block copolymer (PEO)₂(PPO)₄₀(PEO)₂; or combinations thereof.

The capsule can in general have a film-forming material together with optional materials which can include cooling agents, stabilizing agents, and saliva stimulating materials.

The invention further comprises a kit comprising at least one effective dose of a chemotherapeutic agent encapsulated in a semi-solid matrix that further comprises an inhibitor of ABCG2 wherein the amount of the inhibitor is less than, at or about the critical micelle concentration of the inhibitor and a label specifying a dose regimen.

The invention can also comprise a method of treatment of a subject in need thereof comprising administering to said subject a therapeutically effective amount of a pharmaceutically active agent in combination with an inhibitor of ABCG2 wherein the amount of the inhibitor is less than or about the critical micelle concentration of the inhibitor when delivered to a gastrointestinal tract of the subject.

### BRIEF DESCRIPTION OF THE FIGURES

FIG. 1 illustrates the effect of GF 120918 or a dose range of three excipients on [³H]-mitoxantrone uptake in control (GFP, green fluorescent protein) and ABCG2-transduced MDCK-II cells.

FIG. 2 illustrates the effect of various pharmaceutical excipients on [³H]-estrone-3-sulfate (E1S) uptake in HEK vesicles.

FIG. 3 illustrates a measurement of critical micelle concentration for lauryl polyethylene glycol ether.

FIG. 4 illustrates the dose response of six excipients on E1S uptake in HEK vesicles.

FIG. 5 illustrates a data transform of the dose response of six excipients on E1S uptake.

FIG. 6 illustrates the effects of selected excipients on mitoxantrone accumulation in BCRP MDCK-II cells. Results are expressed as mean±SE for n=6.

FIG. 7 illustrates the effect of 15 selected excipients on mitoxantrone accumulation in GFP MDCKII and BCRP MDCKII cells. The results are expressed as means±SE, where n is 3-6. Statistically significant differences in comparison to controls are marked by * (p<0.05) or ** (p<0.01)

FIG. 8 illustrates the effect of 15 selected excipients on mitoxantrone accumulation in GFP MDCKII and P-gp MDCKII cells. The results are expressed as means±SE, where n is 3-6. Statistically significant differences in comparison to controls are marked by * (p<0.05) or ** (p<0.01).

FIG. 9 illustrates the effect of knock-out (KO), i.e., genetic deletion, of the bcrp1 gene on the time course of drug levels in the plasma after administration of topotecan. Results are expressed as means for n=2.

FIG. 10 illustrates the effects of an excipient on the time course of plasma levels of topotecan given orally. Results are expressed as means±SD for n=3. Means that are significantly different from controls are marked with * for n=3. The mice are a) bcrp1 KO and b) wild type.

FIG. 11 illustrates the effect of an excipient on the time course of plasma levels of topotecan after intra-venous administration. Results are expressed as means±SD for n=3. Panel a) shows administration of topotecan to bcrp1 KO mice. Panel b) shows administration of topotecan to wild type mice.

### DETAILED DESCRIPTION OF THE INVENTION

### DEFINITIONS

In this application, the following terms are used according to the following meanings:

A “substrate” of ABCG2 is the molecule that the active transporter transports. Some of the known substrates of ABCG2 are anthracyclines, mitoxantrone, bisantrene, camptothecins (including topotecan and the metabolite SN-38), prazosin, doxorubicin, glucuronide conjugates (including E217βG, 4-methylumbelliferone glucuronide, and E3040 glucuronide), and sulfate conjugates (including estrone sulfate, estradiol sulfate, DHEAS, 4-methylumbelliferone sulfate and E3040 sulfate).

“Xenobiotic” is a chemical compound or biological compound that is foreign to the body of a particular living organism. Pesticides and synthetic or semi-synthetic drugs exemplify xenobiotics.

“Active excipients” are those able to affect drug absorption, in particular, those excipients that inhibit ABCG2 function.

“Inert excipients” are excipients other than active excipients.

“Active pharmaceutical agent” is the primary drug administered to treat disease.

The unit for measuring excipient concentration is molar (M), and related units at other concentrations, such as micromolar (μM or uM).

The critical micelle concentration can be measured by a surface tension method,-e.g. using a Tensiometer, or other methods known in the art. Any suitable method known in the art can be used to measure the cmc. In a particular embodiment ASTM D 971 REV A is used.

Nomenclature of genes and gene products is as follows and reflects lingering use of non-systematic names. A gene name is written in lowercase italics unless derived from a proper name and the gene expression product is often all uppercase and not italicized. Thus, the gene encoding the human breast cancer resistance protein is bcrp (also known as abcg2) and the gene expression product is ABCG2. The chromosomal locus of the gene is 4q22. In mice, the gene bcrp1 encodes bcrp1. By comparison the human multi-drug resistance gene is mdr1 (also known as abcb1) and encodes the P-glycoprotein (P-gp) also known as ABCB1. Inhibitors of ABCG2 and/or the murine homologue include reserpine, CI 1033, GF 120918, fumitremorgin C (FTC), Ko 134 and Ko 132.

In particular aspects, the methods and formulations of the invention are directed to a particular active excipient. In one particular aspect of the invention the active excipient is polyoxyl 35 castor oil (e.g. Cremophor EL). In another particular aspect of the invention, the active excipient is polyoxyethylenesorbitan monolaurate (&.g. Tween 20). In yet another particular aspect of the invention the active excipient is lauryl polyethylene glycol ether (e.g. Bri130). In still another particular aspect of the invention the active excipient is ethylene oxide/propylene oxide block copolymer; (PEO)₂₆(PPO)_{39.5}(PEO)₂₆ (e.g. Pluronic P85). In still yet another particular aspect of the invention the active excipient is ethylene oxide/propylene oxide block copolymer; (PEO)₂(PPO)₄₀(PEO)₂ (e.g. Pluronic L81). In a particular aspect the active excipient is polysorbate 80 (e.g. Tween 80). In another particular aspect the active excipient is LM. In yet another particular aspect, the active excipient is polyoxyl 40 stearate (e.g. Myr152). In still another particular aspect the active excipient is polyoxyethyleneglyceroltrihydroxystearate (e.g. Cremophor RH 40). In yet still another particular aspect the active excipient is Vitamin E TPGS. See Table 1 for a further chemical description of these and other excipients.

The amount of active excipient in the compositions and methods of the invention can vary. In particular aspects of the invention the amount of the excipient is viewed with relation to the value of the cmc. For example, the amount can be one-twentieth of the cmc, one tenth of the cmc, one fifth of the cmc and so forth. As another example, the amount can be two, five, ten, thirty or one hundred times the cmc, or intermediate values. Even more particular values can include ranges such as about one-twentieth to about one-fifth of the cmc; 2-100 times the cmc; 10-100 times the cmc; 2-30 times the cmc; 5-30 times the cmc; and 10-30 times the cmc. Moreover, the amount can be related to dispersion of the excipient in the gut or in suitable model systems known in the art. In this way, the amount formulated in, for example, a capsule, can be related to the concentration, relative to the cmc, resulting from administration. In a particular embodiment, the amount of excipient which is administered is determined such that when diluted into the stomach or intestinal fluid, the concentration is less than the cmc of the excipient. The volumes of the upper gastrointestinal tract can be approximated as follows: fasted state stomach, 300-500 ml; fed state stomach, 900ml; fasted state small intestine, 500 ml; fed state small intestine, 900-1000 ml; and fasted state stomach plus coadministered fluid, 50 ml. Dressman and Reppas, 2000, In Vitro-In Vivo Correlations for Lipophilic, Poorly Water-soluble Drugs, Eur. J. Pharm. Sci. 11 Supp 2, S73. In a conventional method the volume is taken to correspond to a glass of water, about 200 ml. In another embodiment the amount is based on the volume of the fluid in duodenum, and/or jejunum and/or ileum.

The amount of active excipient which is administered can be determined by measurement of the critical micelle concentration. The critical micelle concentration can be measured upon dilution of the active excipient in any suitable fluid, including, but not limited to, water, deuterated water, an aqueous buffered solution, a buffered or unbuffered saline solution, a natural stomach fluid, a simulated stomach fluid, a natural intestinal fluid, or a simulated intestinal fluid. The natural and simulated stomach and intestinal fluids can be from the unfed or fed state. Exemplary simulated media are provided by Dressman and Reppas, Id.; and Galia et al., 1998, Evaluation of Various Dissolution Media for Predicting In Vivo Performance of Class I and II Drugs,Pharm. Res. 15, 698. A suitable fluid is fasted state simulated intestinal fluid (FaSSIF). Another suitable fluid is fed state simulated intestinal fluid (FeSSIF). Exemplary Formulations of FaSSIF and FeSSIF are provided in Table 1.

TABLE 1

| Component | FaSSIF | Component | FeSSIF |
|---|---|---|---|
| Sodium | 3 mM | Sodium | 15 mM |
| taurocholate | | taurocholate | |
| Lecithin | 0.75 mM | Lecithin | 3.75 mM |
| NaOH (pellets) | 0.174 g | NaOH (pellets) | 4.04 g |
| NaH₂PO₄H₂O | 1.977 g | Acetic acid, glacial | 8.65 g |
| NaCl | 3.093 g | NaCl | 11.874 g |
| Purified water qs | 500 ml | Purified water qs | 1000 ml |
| pH | 6.5 | pH | 5.0 |

In addition to the above factors, one of skill in the art can consider additional factors in correlating in vitro measurements with in vivo effect. Such factors can include, but are not limited to, temperature, the absence or presence of enzymes such as lipases, and body size of the subject.

Any method known in the art may be used to measure the critical micelle concentration including, but not limited to, surface tension measurements, fluorescence measurements, and near infrared measurements. See, e.g., Tran and Yu, 2005, Near Infrared Spectroscopic Method for the Sensitive and Direct Detennination of Aggregations of Surfactants in Various Media, J. Colloid Interface Sci. 283, 613.

Administration of the compounds of the invention, in an appropriate pharmaceutical composition, can be carried out via any of the accepted modes of administration to mucosal membranes. Thus, administration can be, for example, orally, nasally, topically, vaginally, bucally, rectally, or to the lungs or bronchii, in the form of solid, semi-solid, lyophilized powder, or liquid dosage forms, such as for example, tablets, suppositories, pills, soft elastic and hard gelatin capsules, powders, solutions, suspensions, or aerosols, or the like, in particular aspects in unit dosage forms suitable for simple administration of precise dosages. The compositions will include a conventional pharmaceutical carrier, the active excipient of the invention an active pharmaceutical agent, and, in addition, may include other medicinal agents, pharmaceutical agents, carriers, adjuvants, etc. The “inert” excipients can include, for example, pharmaceutical grades of mannitol, lactose, starch, pregelatinized starch, magnesium stearate, sodium saccharine, talcum, cellulose ether derivatives, glucose, gelatin, sucrose, citrate, propyl gallate, dicalcium phosphate, and the like; a disintegrant such as croscarmellose sodium or derivatives thereof; a lubricant such as magnesium stearate and the like; and a binder such as a starch, gum acacia, polyvinylpyrrolidone, gelatin, cellulose ether derivatives, and the like. Such compositions take the form of solutions, suspensions, tablets, pills, capsules, powders, sustained release formulations and the like.

For oral administration, formulations of the invention may be administered in nutritionally accepted vehicles for oral ingestion, such as, capsules, tablets, or pills, soft gel caps, powders, solutions, dispersions, or liquids. In preparing the compositions in oral dosage form, any of the usual media may be employed. For oral liquid preparations (e.g., suspensions, elixirs, and solutions), media containing, for example, water, oils, alcohols, flavoring agents, preservatives, coloring agents and the like may be used. Carriers such as starches, sugars, diluents, granulating agents, lubricants, binders, disintegrating agents, and the like may be used to prepare oral solids (e.g., powders, capsules, pills, tablets, and lozenges). Controlled release forms may also be used. A tablet may be made by compression or molding, optionally with one or more accessory ingredients. Compressed tablets may be prepared by compressing in a suitable machine the active ingredient in a free-flowing form such as a powder or granules, optionally mixed with a binder (e.g. Povidone, which is 1-ethenylpyrrolidin-2-one, gelatin, or hydroxypropylmethylcellulose), lubricant, inert diluent, preservative, disintegrant (e.g. sodium starch glycollate, cross-linked Povidone, cross-linked sodium carboxymethylcellulose) surface-active agent or dispersing agent. Molded tablets may be made by molding in a suitable machine a mixture of the powdered compound moistened with an inert liquid diluent. The tablets may optionally be coated or scored and may be formulated so as to provide controlled release of the active ingredients therein using, for example, hydroxypropylmethylcellulose in varying proportions to provide the desired release profile. Soft gelcaps are particular exemplifications for containing lipophilic substances, such as tocopherols and polyunsaturated fatty acids. Methods for preparing gelcaps are well known in the art. See, for example, US2005/0152971 which discloses a gelcap with an exposed circumferential band; U.S. Pat. No. 5,317,849 which discloses a soft gelatin coated tablet core; U.S. Pat. Nos. 5,089,270 and 5,213,738 directed to a clear gelatin coating on a colored tablet; and U.S. Pat. Nos. 4,820,524, 4,966,771 and 4,867,983 directed to a gelatin coated tablet core.

The oral dosage form can comprise a semi-solid matrix which optionally further comprises a lecithin. The oral dosage form can also be a semi-solid matrix which comprises a polyglycolized glyceride and, optionally, further comprises a lecithin.

A tablet can be produced by adding, for example, “inert” excipients (e.g., lactose, sucrose, starch, D-mannitol etc.), disintegrants (e.g., carboxymethyl cellulose calcium etc.), binders (e.g., pregelatinized starch, gum arabic, carboxymethyl cellulose, hydroxypropyl cellulose, polyvinylpyrrolidone etc.), lubricants (e.g., talc, magnesium stearate, polyethylene glycol 6000 etc.), an “active” excipient (e.g. polyoxyl 35 castor oil, polyoxyl 4 lauryl ether, polyoxyethylenesorbitan monolaurate, etc.) and the like, to the active ingredient, compression-shaping, and, where necessary, applying a coating by a method known per se using a coating base known per se for the purpose of achieving taste masking, enteric dissolution or sustained release.

The capsule can be a gelatin capsule or a polysaccharide capsule such as a cellulose capsule. Any gelatin known by one of skill in the art to be suitable for preparation of capsules can be used to form the gelatin capsules, including, but not limited to, bovine gelatin, porcine gelatin, fish gelatin, and pure isinglass. In a cellulose capsule the film-forming material can be a cellulosic polymer, including, but not limited to, hydroxypropyl cellulose, hydroxyethyl cellulose, hydroxypropyl methyl cellulose, hydroxymethyl cellulose, methyl cellulose, ethyl cellulose, cellulose acetate, cellulose acetate phthalate, cellulose acetate trimelliate, hydroxypropylmethyl cellulose phthalate, hydroxypropylmethyl cellulose succinate, carboxymethyl cellulose sodium, and mixtures thereof. The capsule can also be formed from pullulan or other glucans such as scleroglucan, polyvinyl alcohol, pectin, modified starches, alginates including sodium, ammonium, potassium, or calcium alginate, or propylene alginate, polyvinyl pyrrolidone, carboxyvinyl polymer, polyacrylic acid, soligel, chitin, chitosan, levan, elsinan, gelatin, collagen, zein, gluten, soy protein isolate, whey protein isolate, casein, or gums including xanthan gum, tragacanth gum, guar gum, acacia gum, Arabic gum, locust bean gum, and gum ghatti. The modified starches can, in particular, be starch ethers or oxidized starch and more particularly hydroxypropylated starch or hydroxyethylated starch. The capsule can take any suitable form known in the pharmacological arts. For example, the capsule can be a hard-shell capsule or a soft-shell capsule. In one particular aspect, the capsule can comprise pullulan. In one form, a capsule can be enterically coated. The capsule can also include stabilizing agents including xanthan gum, locust bean gum, guar gum, and carrageenan in amounts ranging from about 0 to about 10 weight %, preferably about 0.1 to about 2 weight % of the film. Such capsules and enteric coatings can include those known in the art such as described in U.S. Pat. No. 6,887,307, directed to pullulan capsules; U.S. Pat. No. 6,849,269, and U.S. Pat. No. 6,761,901 directed to proliposomal delivery systems; U.S. Pat. No. 6,752,953 directed to non-gelatin hard pharmaceutical capsules; U.S. Pat. No. 6,627,219 directed to an oily capsule; U.S. Pat. No. 6,531,152 directed to a capsule for immediate release at a specific gastrointestinal site; U.S. Pat. No. 6,517,865 directed to polymer films suitable for capsules; U.S. Pat. No. 6,455,052 directed to alginic acid coating for capsules and tablets; U.S. Pat. No. 6,331,316 directed to an enteric tablet coating suitable for acid-sensitive drugs; U.S. Pat. No. 6,214,378 directed to a capsule having a cationic polymer coating and an outer anionic polymer coating; and U.S. Pat. No. 5,447,729 directed to multilamellar drug delivery systems.

The capsule can be made as a hard capsule filled with a powder or granular pharmaceutical agent, or a soft capsule filled with a liquid or suspension liquid or a semi-solid matrix. The hard capsule is produced by mixing and/or granulating an active ingredient with, for example, an excipient (e.g., lactose, sucrose, starch, crystalline cellulose, D-mannitol and the like), a disintegrant (e.g., low substituted hydroxypropyl cellulose, carmellose calcium, corn starch, croscarmellose sodium and the like), an “active” excipient (e.g. polyoxyl 35 castor oil, polyoxyl 4 lauryl ether, polyoxyethylenesorbitan monolaurate, etc.), a binder (e.g., hydroxypropyl cellulose, polyvinylpyrrolidone, hydroxypropylmethyl cellulose and the like), a lubricant (e.g., magnesium stearate and the like) and the like, and filling the mixture or granule in a capsule formed from the aforementioned gelatin, pullulan, and the like. The cellulose can be hydroxymethyl cellulose, hydroxypropylmethyl cellulose, or any other form of cellulose known in the art. The soft capsule is produced by dissolving or suspending the active ingredient in a base (e.g., soybean oil, cottonseed oil, medium chain fatty acid triglyceride, beeswax and the like having an “active” excipient (e.g. polyoxyl 35 castor oil, polyoxyl 4 lauryl ether, Polyoxyethylenesorbitan monolaurate, etc.)) and sealing the prepared solution or suspension in a gelatin sheet using, for example, a rotary filling machine and the like.

Conventional hard capsules are made with gelatin by a dip molding process. The dip molding process is based on the ability of hot gelatin solutions to set by cooling. For the industrial manufacture of pharmaceutical capsules, gelatin is preferred for its gelling, film forming and surface active properties. A typical dip molding process comprises the steps of dipping mold pins into a hot solution of gelatin, removing the pins from the gelatin solution, allowing the gelatin solution attached on pins to set by cooling, drying and stripping the so-formed shells from the pins. The setting of the solution on the mold pins after dipping is the critical step to obtain a uniform thickness of the capsule shell. On a totally automatic industrial hard gelatin capsule machine, the pins having a coating of gelatin are turned from downside to upside to dry. the gelatin solution attached on the pins. When the gelatin is cool and set, the capsule shell is stripped from the pin and subsequently cut and the cap and body are joined. The rapid setting of the gelatin solution on the dip pins after dipping is important in maintaining uniform wall thickness.

U.S. Pat. No. 2,526,683 to Murphy first described a process for preparing methyl cellulose medicinal capsules by a dip coating or dip molding process. The process consists of dipping a capsule forming pin pre-heated to 40-85° C. into a cellulose ether solution kept at a temperature below the incipient gelation temperature, withdrawing the pins at a predetermined withdrawal speed and then placing the pins in ovens kept at temperatures above the gelation temperature, exposing the pins to a lower temperature first and then gradually to higher temperature until the film is dry. The dry capsule is then stripped, cut to size, and the body and caps are fitted together. These methylcellulose capsules, however, fail to dissolve in the gastrointestinal fluid at body temperature in an acceptable time.

Sarkar's U.S. Pat. No. 4,001,211 describes a medicinal capsule using thermal gelling cellulose ethers such as methyl cellulose and hydroxypropylmethyl cellulose. Sarkar's capsules are prepared by a pin dip coating process by blending water soluble methyl and C₂-C₃ hydroxyalkyl cellulose ethers to achieve an essentially Newtonian dip coating solution. Blends of low viscosity methyl cellulose and hydroxypropylmethyl cellulose provide particularly suitable dip solution properties, gel yield strength, and capsule dissolution rates.

Muto's U.S. Pat. No. 4,993,137 is directed to the manufacture of capsules made from the improved methyl cellulose ether of Sarkar. Muto discloses a process for gelling the solution by dipping solution coated pins into thermally controlled water.

Grosswald et al.'s U.S. Pat. No. 5,698,155 describes a method and apparatus to manufacture pharmaceutical capsules. The method uses an aqueous solution of a thermogelling cellulose ether composition with capsule body pins and capsule cap pins as molds. The method further involves heating the pins, dipping the pins into the cellulose-containing aqueous solution to cause the solution to set on the surface of the pins, removing the pins, and drying the coated pins to form the capsule bodies and capsule caps.

Capsules and other dosage delivery devices can be made from pullulan. Pullulan is a natural, viscous, water-soluble polysaccharide, which is be produced extracellularly by growing certain yeasts on starch syrups. It has good film forming properties, a particularly low oxygen permeability, and a moisture content at 50% RH of about 12%. U.S. Pat. No. 4,623,394 describes a molded capsule, consisting essentially of a combination of pullulan and a heteromannan, which has a controlled rate of disintegration under hydrous conditions. JP5-65222-A describes a soft capsule, capable of stabilizing a readily oxidizable substance enclosed therein, exhibiting easy solubility, and being able to withstand a punching production method. The soft capsule is obtained by blending a capsule film substrate such as gelatin, agar, or carrageenan with pullulan. U.S. Pat. No. 3,784,390 discloses that certain mixtures of pullulan with amylose, polyvinyl alcohol, or gelatin can be shaped by compression molding or extrusion at elevated temperatures or by evaporation of water from its aqueous solutions to form shaped bodies, such as films or coatings. U.S. Pat. No. 4,562,020, discloses a continuous process for producing a self-supporting glucan film, such as pullulan or elsinan; comprising casting an aqueous glucan solution on the surface of a corona-treated endless heat-resistant plastic belt, drying the glucan solution on the belt, and releasing the resultant self-supporting glucan film. JP-60084215-A2 discloses a film coating composition for a solid pharmaceutical having improved adhesive properties on the solid agent. The film is obtained by incorporating pullulan with a film coating base material such as methylcellulose. JP-2000205-A2 discloses a perfume-containing coating for a soft capsule. The coating is obtained by adding a polyhydric alcohol to a pullulan solution containing an oily perfume and a surfactant such as a sugar ester having a high HLB. U.S. Pat. No. 3,871,892 describes the preparation of pullulan esters by the reaction of pullulan with aliphatic or aromatic fatty acids or their derivatives in the presence of suitable solvents and/or catalysts. The pullulan esters can be shaped by compression molding or extrusion at elevated temperatures or by evaporation of solvents from their solutions to form shaped bodies such as films or coatings. U.S. Pat. No. 3,873,333 discloses adhesives or pastes prepared by dissolving or dispersing uniformly a pullulan ester and/or ether in water or in a mixture of water and acetone in an amount between 5 percent and 40 percent of the solvent. U.S. Pat. No. 3,997,703 discloses a multilayered molded plastic having a pullulan layer and a layer composed of homopolymers and copolymers of olefins and/or vinyl compounds, polyesters, polyamides, celluloses, polyvinylalcohol, rubber hydrochlorides, paper, or aluminum foil. GB 1,533,301 describes a method of improving the water-resistance of pullulan by the addition of water-soluble dialdehyde polysaccharides to pullulan. GB 1559 644 also describes a method of improving the water-resistance of pullulan articles. The improved articles are manufactured by means of a process comprising bringing a mixture or shaped composition of a (a) pullulan or a water soluble derivative thereof and (b) polyuronide or a water-soluble salt thereof in contact with an aqueous and/or alcoholic solution of a di- or polyvalent metallic ion. WO 01/07507 generally describes pullulan film compositions and setting systems. US2005/0249676 discloses addition of a setting system to a pullulan solution to facilitate production of hard capsules using a dip molding process.

Yamamoto et al.'s U.S. Pat. No. 5,756,123 discloses a capsule shell containing 79.6-98.7% by weight of a hydroxypropylmethyl cellulose (HPMC) as a water-soluble cellulose derivative base, 0.03-0.5% by weight of carrageenan as a gelling agent, and 0.14-3.19% by weight of a potassium ion and/or a calcium as a co-gelling agent. The capsule shell is prepared by blending the HPMC with carrageenan in the water to form an aqueous solution, and drying the aqueous solution to form a capsule shell using the conventional immersion molding method.

US2003/0104047 discloses a method for manufacturing hard non-gelatin capsules by a heat melting method to melt the capsule forming composition in a mold. The capsule shell is formed after a pre-heated pestle is inserted into the mold. The pressure applied by the pestle ensures that the melted capsule forming composition is evenly coated onto the pestle. The pestle is then retrieved from the mold, taking the coated capsule forming composition with it, which is subsequently dried and removed from the pestle to become the capsule shell. The method does not require preparation of an aqueous capsule forming composition, which saves time and may be cost-effective compared to the dip molding method.

US2004/0265384 discloses a composition for formation of soluble films comprising partially hydrolyzed exopolysaccharide YAS34 from Rhizobium Leguninasorum. The polysaccharide is also known as Soligel. The '384 application adds an additional setting agent to YAS34 to improve the working temperature during manufacture.

US2005/0196437 discloses a blend of a physically induced starch hydrolysate, a plasticizer, and a gelling agent which has a film with a high modulus and excellent toughness, for manufacture of gelatin-free hard capsules.

The subject formulations may be compounded with physiologically acceptable materials which can be ingested including, but not limited to, foods, including, but not limited to, food bars, beverages, powders, cereals, cooked foods, food additives and candies. When the composition is incorporated into various media such as foods, it may simply be orally ingested. The food can be a dietary supplement (such as a snack or wellness dietary supplement) or, especially for animals, comprise the nutritional bulk (e.g., when incorporated into the primary animal feed). The subject to whom the pharmaceutical agent is administered can be a human, although veterinary use is also specifically contemplated.

For rectal administration, the subject compositions may be provided as suppositories, as solutions for enemas, or other convenient application. Suppositories may have a suitable base comprising, for example, cocoa butter or a salicylate. Formulations for vaginal administration may be presented as pessaries, tampons, creams, gels, pastes, foams or spray formulations containing in addition to the active ingredient such carriers as are known in the art to be appropriate.

Generally, depending on the intended mode of administration, the pharmaceutically acceptable compositions will contain about 1% to about 99% by weight of an ABCG2 inhibitor, from 1% to about 99% by weight of an active pharmaceutical agent, and 99% to 1% by weight of a suitable “inert” pharmaceutical excipient. In a particular example, the composition will be about 5% to 75% by weight of an active pharmaceutical agent, or a pharmaceutically acceptable salt thereof, with the rest being suitable pharmaceutical excipients, including active excipients that inhibit ABCG2. In another particular example the active excipient is less than 50% by weight of the composition, with the weights being based on the total about of the composition.

Liquid pharmaceutically administrable compositions can, for example, be prepared by dissolving, dispersing, etc., an active pharmaceutical agent (about 0.5% to about 20%), or a pharmaceutically acceptable salt thereof, and pharmaceutical adjuvants including the active excipients of the invention in a carrier, such as, for example, water, saline, aqueous dextrose, glycerol, ethanol and the like, to thereby form a solution or suspension.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Actual methods of preparing such dosage forms are known, or will be apparent, to those skilled in this art; for example, see Remington's Pharmaceutical Sciences, 20th Ed., (Mack Publishing Company, Easton, Pa., 2000). The composition to be administered will, in any event, contain a therapeutically effective amount of an active agent or prodrug, or a pharmaceutically acceptable salt thereof, for treatment of a disease.

As used herein, as the pharmacologically acceptable carrier, various organic or inorganic carrier substances conventionally used as materials for preparations can be used. For example, excipient, lubricant, binder and disintegrant for solid preparations; solvent, dissolution aids, suspending agent, isotonizing agent and buffer for liquid preparations; and the like can be mentioned. Where necessary, additives for preparation, such as preservative, antioxidant, coloring agent, sweetening agent and the like, can be also used.

Particular examples of an “inert” excipient include lactose, sucrose, D-mannitol, D-sorbitol, starch, pregelatinized starch, dextrin, crystalline cellulose, low-substituted hydroxypropyl cellulose, carboxymethyl cellulose sodium, gum arabic, pullulan, light silicic anhydride, synthetic aluminum silicate, magnesium aluminometasilicate and the like.

Particular examples of a lubricant include magnesium stearate, calcium stearate, talc, colloidal silica and the like.

Particular examples of a binder include pregelatinized starch, sucrose, gelatin, gum arabic, methyl cellulose, carboxymethyl cellulose, carboxymethyl cellulose sodium, crystalline cellulose, sucrose, D-mannitol, trehalose, dextrin, pullulan, hydroxypropyl cellulose, hydroxypropylmethyl cellulose, polyvinylpyrrolidone and the like.

Particular examples of a disintegrant include lactose, sucrose, starch, carboxymethyl cellulose, carboxymethyl cellulose calcium, croscarmellose sodium, carboxymethyl starch sodium, light silicic anhydride, low-substituted hydroxypropyl cellulose and the like.

Particular examples of a solvent include water for injection, physiological brine, Ringer's solution, alcohol, propylene glycol, polyethylene glycol, sesame oil, corn oil, olive oil, cottonseed oil and the like.

Particular examples of dissolution aids include polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like.

Particular examples of a suspending agent include surfactants such as stearyltriethanolamine, sodium lauryl sulfate, lauryl aminopropionate, lecithin, benzalkonium chloride, benzethonium chloride, glycerol monostearate etc.; hydrophilic polymers such as polyvinyl alcohol, polyvinylpyrrolidone, carboxymethylcellulose sodium, ethylcellulose, hydroxymethylcellulose, hydroxyethylcellulose, hydroxypropylcellulose etc.; polysorbates, polyoxyethylene hydrogenated castor oil and the like.

Particular examples of an isotonizing agent include sodium chloride, glycerin, D-mannitol, D-sorbitol, glucose and the like.

Particular examples of a buffer include buffers such as phosphate, acetate, carbonate, citrate etc., and the like.

Particular examples of a preservative include p-oxybenzoate, chlorobutanol, benzyl alcohol, phenethyl alcohol, dehydroacetic acid, sorbic acid and the like.

Particular examples of an antioxidant include sulfite, ascorbate and the like.

Particular examples of a coloring agent include water-soluble edible tar dyes (e.g., food colors such as Food Red Nos. 2 and 3, Food Yellow Nos. 4 and 5, Food Blue Nos. 1 and 2, etc.), water-insoluble Lake dyes (e.g., aluminum salts of the aforementioned water-soluble edible tar dyes etc.), natural colors (e.g., β-carotene, chlorophyll, iron oxide red etc.) and the like.

Particular examples of a sweetening agent include saccharin sodium, dipotassium glycyrrhizinate, aspartame, acesulfame potassium, sucralose, stevia and the like.

When the active pharmaceutical compound is a salt and avoidance of contact of the compound in the form of a salt with water is preferable, the compound can be dry-mixed with an active excipient and the like to give a hard capsule.

As used herein “enteric coating,” comprises a polymeric material, or materials, which encases the medicament core. A suitable enteric coating, of the present invention, is one which will have no significant dissolution at pH levels below 4.5. Enteric coatings, suitable for the present invention, include enteric coating polymers known in the art, for example, hydroxypropyl methylcellulose phthalate (HPMCP-HP50, USP/NF 220824 HPMCP-HP55, USP/NF type 200731 and HP55S; Shin Etsu Chemical), polyvinyl acetate phthalate (Coateric™, Colorcon Ltd.), polyvinyl acetate phthalate (Sureteric™, Colorcon, Ltd.), and cellulose acetate phthalate (Aquateric™, FMC Corp.) and the like. In one aspect, the enteric coating will use a methacrylic acid copolymer.

The dose of the active pharmaceutical compound is determined in consideration of age, body weight, general health condition, sex, diet, administration time, administration method, clearance rate, combination of drugs, the level of disease for which the patient is under treatment then, and other factors.

While the dose varies depending on the target disease, condition, subject of administration, administration method and the like, for oral administration as a therapeutic agent for essential hypertension in adult, the daily dose of 0.1-100 mg is, in a particular example, administered in a single dose or in 2 or 3 portions.

In addition, because the “active” excipients of the present invention are superior in safety, they can be administered for a long period.

The combination of an active pharmaceutical agent and the “active” excipients of the present invention can be used in combination with pharmaceutical agents such as a therapeutic agent for diabetes, a therapeutic agent for diabetic complications, an anti-hyperlipidemia agent, an anti-arteriosclerotic agent, an anti-hypertensive agent, an anti-obesity agent, a diuretic, an antigout agent, an antithrombotic agent, an anti-inflammatory agent, a chemotherapeutic agent, an immunotherapeutic agent, a therapeutic agent for osteoporosis, an anti-dementia agent, an erectile dysfunction amelioration agent, a therapeutic agent for urinary incontinence/urinary frequency and the like (hereinafter to be abbreviated as a combination drug). On such occasions, the timing of administration of the composition of the present invention and that of the combination drug is not limited, as long as the composition of the present invention and the combination drug are combined. As the mode of such administration, for example, (1) administration of a single preparation obtained by simultaneous formulation of the composition of the present invention and a combination drug, (2) simultaneous administration of two kinds of preparations obtained by separate formulation of the composition of the present invention and a combination drug, by a single administration route, (3) time staggered administration of two kinds of preparations obtained by separate formulation of the composition of the present invention and a combination drug, by the same administration route, (4) simultaneous administration of two kinds of preparations obtained by separate formulation of the composition of the present invention and a combination drug, by different administration routes, (5) time staggered administration of two kinds of preparations obtained by separate formulation of the composition of the present invention and a combination drug, by different administration routes, such as administration in the order of the composition of the present invention and then the combination drug, or administration in a reversed order, and the like can be mentioned. The dose of the combination drug can be appropriately determined based on the dose clinically employed. The mixing ratio of the composition of the present invention and the combination drug can be appropriately selected according to the administration subject, administration route, target disease, condition, combination, and other factors. In cases where the administration subject is human, for example, the combination drug may be used in an amount of 0.01 to 100 parts by weight per part by weight of the compound of the present invention.

The “active” excipients of this invention can be administered in combination with known anti-cancer agents. Such known anti-cancer agents include the following: estrogen receptor modulators, androgen receptor modulators, aromatase inhibitors, retinoid receptor modulators, cytotoxic agents, antiproliferative agents, prenyl-protein transferase inhibitors, HMG-CoA reductase inhibitors, HIV protease inhibitors, reverse transcriptase inhibitors, DNA methyl transferase inhibitors, and other angiogenesis inhibitors. Particular angiogenesis inhibitors are selected from the group consisting of a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-(chloroacetyl-carbamoyl)-fumagillol, thalidomide, angiostatin, troponin-1, and an antibody to vascular endothelial growth factor (VEGF).

Particular estrogen receptor modulators are tamoxifen and raloxifene.

“Estrogen receptor modulators” refers to compounds that interfere or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]-1H-1-benzopyran-3-yl]-phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl)retinamide, and N-4-carboxyphenyl retinamide.

“Cytotoxic agents” refer to compounds which cause cell death primarily by interfering directly with the cell's functioning or inhibit or interfere with cell myosis, including alkylating agents, tumor necrosis factors, intercalators, microtubulin inhibitors, and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-arninedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)]-tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, and 4-demethoxy-3-deamino-3-aziridinyl-4-methylsulphonyl-daunorubicin (see WO 00/50032).

Examples of microtubulin inhibitors include prazosin, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, and BMS188797.

Some examples of topoisomerase inhibitors are topotecan, hycaptamine, irinotecan, rubitecan, 6-ethoxypropionyl-3′,4′-O-exo-benzyli-denechartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H-benzo[de]pyrano[3′,4′:b,7]-indolizino[1,2b]quinoline-10,13(9H,15H-)dione, lurtotecan, 7-[2-(N-isopropylamino)-ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)-ethyl]-N-methylamino]ethyl]-5-[4-hydroxy-3,5-dimethoxyphenyl]-5,5a,6,8,8a,-9-hexohydrofuro(3′,4′,6,7)colchic(2,3-d)-1,3-dioxol-6-one, 2,3-(methylenedioxy)-5-methyl-7-hydroxy-8-methoxybenzo[c]-phenanthridinium, 6,9-bis[(2-aminoethyl)-amino]benzo[g]isoguinoline-5, 10-dione, 5-(3-amninopropylamino)-7,10-dihydroxy-2-(2-hydroxyethylaminomethyl)-6H-pyrazolo[4,5, 1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2-, 1-c]quinolin-7-one, and dimesna.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxy-cytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]-adenine, aplidine, ecteinascidin, troxacitabine, 4-[2-amino-4-oxo-4,6,7,8-tetrahydro-3H-pyrimidino [5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,1-1-diazatetra cyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, and 3-aminopyridine-2-carboxaldehyde thiosemicarbazone.

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein. The combination of lovastatin, a HMG-CoA reductase inhibitor, and butyrate, an inducer of apoptosis can be used for an antitumor effect.

Examples of HMG-CoA reductase inhibitors that may be used include but are not limited to lovastatin (MEVACOR™; see U.S. Pat. Nos. 4,231,938; 4,294,926; 4,319,039), simvastatin (ZOCOR™; see U.S. Pat. Nos. 4,444,784; 4,820,850; 4,916,239), pravastatin (PRAVACHOL™; see U.S. Pat. Nos. 4,346,227; 4,537,85.9; 4,410,629; 5,030,447 and 5,180,589), fluvastatin (LESCOL™; see U.S. Pat. Nos. 5,354,772; 4,911,165; 4,929,437; 5,189,164; 5,118,853; 5,290,946; 5,356,896), atorvastatin (LIPITOR™; see U.S. Pat. Nos. 5,273,995; 4,681,893; 5,489,691; 5,342,952) and cerivastatin (also known as rivastatin and BAYCHOL™; see U.S. Pat. No. 5,177,080). The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity. The use of such salts, esters, open-acid and lactone forms is included within the scope of this invention.

In HMG-CoA reductase inhibitors where an open-acid form can exist, salt and ester forms can, in a particular example, be formed from the open-acid, and all such forms are included within the meaning of the term “HMG-CoA reductase inhibitor” as used herein. In particular, the HMG-COA reductase inhibitor can be selected from lovastatin and simvastatin.

### EXAMPLES

The following Examples are offered as illustrative of the invention and are not to be construed as limitations thereon. In the Examples and elsewhere in the description of the invention, chemical symbols and terminology have their usual and customary meanings. The term comprising shall be read as including the subgroups of consisting and consisting essentially of. In the Examples as elsewhere in this application values for formulas, molecular weights and degree of ethoxylation or propoxylation are averages. Temperatures are in degrees C. unless otherwise indicated. The amounts of the components are in weight percents based on the standard described; if no other standard is described then the total weight of the composition is to be inferred. It will be understood that numerous additional formulations can be prepared without departing from the spirit and scope of the present invention.

### Example 1

### Effect of Excipients in ABCG2-Transduced Cells

Methods: For constructing MDCK-II cells expressing human ABCG2 or green fluorescent protein (GFP), MDCK-II cells were infected with recombinant adenoviruses containing human ABCG2 or GFP cDNA at 48 h prior to the experiments. ABCG2 or GFP-transduced cells were preincubated in prewarmed transport buffer for 15 min. Subsequently, [³H]-mitoxantrone (MTX) was added in transport buffer to apical compartments. Accumulation of radiolabeled substrates was allowed for 2 h at 37° C. with or without an appropriate concentration of pharmaceutical excipients, 20 μM of GF120918 or 5 μM of PSC833. The reactions were arrested by washing cells with ice-cold transport buffer. Cells were solubilized and then the lysates were transferred to a liquid scintillation counter for measurement of radioactivity.

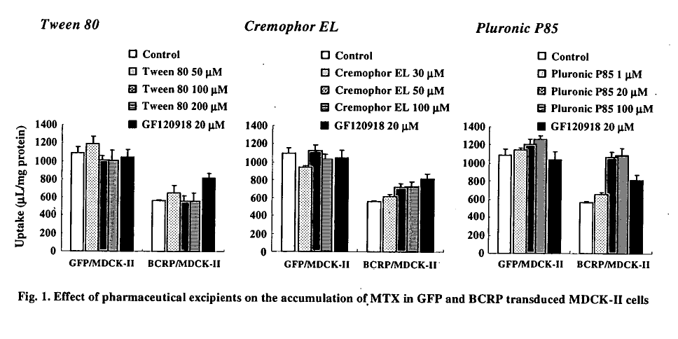

Results: The effect of pharmaceutical excipients, such as polyoxyethylenesorbitan monooleate, polyoxyl 35 castor oil and ethylene oxide/propylene oxide block copolymer; (PEO)₂₆(PPO)_{39.5}(PEO)₂₆, on the accumulation of MTX in GFP and ABCG2 transduced MDCK-H cells is shown in FIG. 1. To abolish the influence of endogenous P-gp, PSC833 (P-gp inhibitor) was added during the incubation for 2 h, except that in the samples treated with GF120918 (a shared ABCG2 and P-gp inhibitor) no PSC833 was added.

GF120918 markedly increased the accumulation of MTX in ABCG2-transduced cells (1.4 times compared to control) while no consistent effect was observed on GFP-transduced cells. Polyoxyethylenesorbitan monooleate did not increase the accumulation of MTX in ABCG2-transduced cells at any concentration. In contrast, polyoxyl 35 castor oil increased the accumulation 1.3 times compared to control in ABCG2-transduced cells at 50 μM. Moreover, ethylene oxide/propylene oxide block copolymer; (PEO)₂₆(PPO)_{39.5}(PEO)₂₆ also significantly enhanced the accumulation 1.9 times compared to control in ABCG2-transduced cells at 20 and 100 μM. Therefore, these results clearly indicate that polyoxyl 35 castor oil and ethylene oxide/propylene oxide block copolymer; (PEO)₂₆(PPO)_{39.5}(PEO)₂₆ significantly inhibit ABCG2 function.

### Example 2

### Inhibitory Effect of the Pharnaceutical Excipients on ABCG2 Function

Measurement of the uptake of [³H]-estrone-3-sulfate (E1S) was carried out for 11 pharmaceutical excipients at or near their reported critical micelle concentrations using ABCG2-expressing membrane vesicles.

Eleven of the pharmaceutical excipients of Table 2 were examined for effect on ABCG2 function at their cmc. The inhibitory mechanism of ABCG2 inhibition by pharmaceutical excipients was also investigated. Membrane vesicles were prepared from HEK293 cells, which had a high level of ABCG2 expression. The uptake of E1S into these membrane vesicles was examined in the presence and absence of the first eleven excipients of Table 2.

TABLE 2

| Chemical Name | Generic Description | Trade Name |
|---|---|---|
| Polyoxyethyleneglyceroltri- | Polyoxyethylene Castor | Cremophor EL |
| ricinoleate 35 | oil | |
| Polyoxyethylenesorbitan | Polyoxyethylene Sorbitan | Tween 80 |
| monooleate | Fatty Acid Esters | |
| | (Polysorbate 80) | |
| Polyoxyethylenesorbitan | Polyoxyethylene Sorbitan | Tween 20 |
| monolaurate | Fatty Acid Esters | |
| | (Polysorbate 20) | |
| Lauryl polyethylene glycol | Polyoxyethylene Alkyl | Brij 30 |
| ether | Ethers | |
| n-dodecyl-β-D- | | n-dodecyl-b-D- |
| maltopyranoside | | maltopyranoside |
| | | (LM) |
| Polyoxyethylene 40 | Polyoxyethylene | Myrj 52 |
| stearate | Stearates | |
| Polyoxyethyleneglyceroltri- | Polyoxyethylene Castor | Cremophor |
| hydroxystearate | oil | RH 40 |
| D-α-tocopheryl | Alpha Tocopherol | Vitamin E TPGS |
| polyethylene glycol 1000 | | |
| succinate | | |
| Ethylene Oxide/Propylene | Poloxamer | Pluronic P85 |
| Oxide Block Copolymer; | | |
| (PEO)₂₆(PPO)_{39.5}(PEO)₂₆ | | |
| Ethylene Oxide/Propylene | Poloxamer | Pluronic L81 |
| Oxide Block Copolymer; | | |
| (PEO)₂(PPO)₄₀(PEO)₂ | | |
| Ethylene Oxide/Propylene | Poloxamer 188 | Pluronic F68 |
| Oxide Block Copolymer; | | |
| (PEO)₁₉(PPO)₁₇(PEO)₁₉ | | |
| Sorbitan monolaurate | | Span 20 |
| Sorbitan monopalmitate | | Span 40 |
| Sorbitan monooleate | | Span 80 |
| PEG-32 glyceryl laurate | | Gelucire 44/14 |

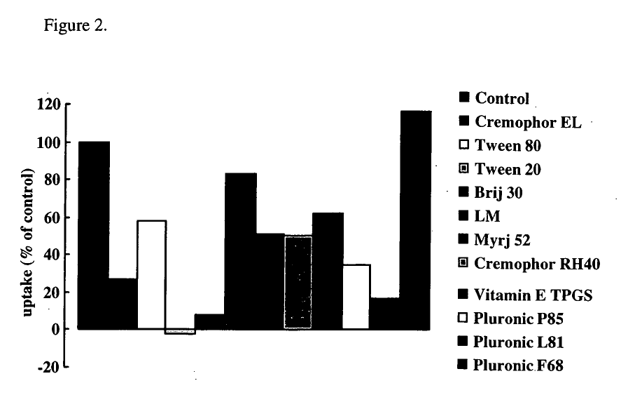

The effect of the “active” excipients of ABCG2 inhibition was observed at concentrations close to the cmc. Table 3 shows the reported cmc value of each of the excipients and measured cmc values. The experimental results are shown in FIG. 2. Polyoxyl 35 castor oil, polyoxyethylenesorbitan monolaurate, polyoxyl 4 lauryl ether, ethylene oxide/propylene oxide block copolymer; (PEO)₂₆(PPO)_{39.5}(PEO)₂₆ and ethylene oxide/propylene oxide block copolymer; (PEO)₂(PPO)₄₀(PEO)₂ decreased the ABCG2-mediated uptake of E1S to less than 40% of control. Polyoxyethylenesorbitan monooleate, polyoxyl 40 stearate, polyoxyethyleneglyceroltrihydroxystearate and vitamin E TPGS inhibited by 40-70%. LM and poloxamer 188 had less or no effect. These results suggest that almost all the tested excipients had an inhibitory effect on ABCG2. Poloxamer 188 affected the function of neither the P-gp nor the ABCG2 transporter. Based on the results of this screening test, we categorize excipients as weak inhibition (uptake >40%) and strong inhibition (uptake <40%).

Thus, the uptake of [³H]-estrone-3-sulfate (E1S) was measured in the presence of 11 pharmaceutical excipients at around their reported critical micelle concentration (cmc) using ABCG2-expressing membrane vesicles. Ten out of the 11 excipients, that is, all except poloxamer 188, decreased uptake of E1S. This decrease was particularly noticeable (uptake became not greater than 40%) with respect to polyoxyl 35 castor oil, polyoxyethylenesorbitan monolaurate, polyoxyl 4 lauryl ether, ethylene oxide/propylene oxide block copolymer; (PEO)₂₆(PPO)_{39.5}(PEO)₂₆ and ethylene oxide/propylene oxide block copolymer; (PEO)₂(PPO)₄₀(PEO)₂.

### Example 3

### Measurement of the CMC OF Pharmaceutical Excipients

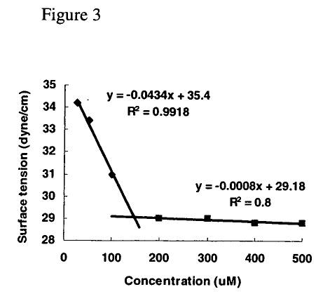

The studies of vesicles revealed that the cmc was an important factor. Therefore, we determined the cmc of the 11 excipients used above, and others, by measuring surface tension in a transport buffer. FIG. 3 shows the exemplary effect of polyoxyl 4 lauryl ether at different concentrations on the surface tension. The concentration beyond which there was no further change in surface tension was taken as the cmc. Table 3 shows the cmc of the excipients determined by measuring the surface tension as in FIG. 3, and the cmc reported in literature, which we have used as reference values.

TABLE 3

| | Measured cmc | Reference |
|---|---|---|
| Excipient | (μM) | cmc (μM) |
| Polyoxyethylenesorbitan | 261 | 270 |
| monolaurate | | |
| Polyoxyl 4 lauryl ether | 146 | 360 |
| Polyoxyl 35 castor oil | 24 | 30 |
| Poloxamer 188 | 222 | 480 |
| Poloxamer (Ethylene | 21 | 65 |
| Oxide/Propylene Oxide | | |
| Block Copolymer; | | |
| (PEO)₂(PPO)₄₀(PEO)₂) | | |
| Poloxamer (Ethylene | 6 | 23 |
| Oxide/Propylene Oxide | | |
| Block Copolymer; | | |
| (PEO)₁₉(PPO)₁₇(PEO)₁₉) | | |
| LM | 688 | 170 |
| PEG 300 | n.d. | n.d. |
| Polyoxyl 40 stearate | 278 | 310 |
| Polyoxyl 40 hydrogenated | 65 | 90 |
| castor oil | | |
| Vitamin E TPGS | 160 | 132 |
| Polyoxyethylenesorbitan | 110 | 50-80 |
| monooleate | | |
| Sorbitan monolaurate | | 100 |
| Sorbitan monopalmitate | | 200 |
| Sorbitan monooleate | | 100 |
| PEG-32 glyceryl laurate | | 15 |
| Propylene glycol | n.d. | |
| Glyceryl triacetate | n.d. | |
| Ethyl oleage | n.d. | |

In the table, n.d. means not determined.

Although there were slight disparities, the measured cmc values were in general agreement with the reference values reported in literature. For LM, however, we found an appreciably higher value from the one reported in the literature.

### Example 4

### IC₅₀ and Hill Coefficients of Selected Excipients

Based on the results obtained in Example 2, six excipients were selected for determining the IC₅₀ and cooperativity of inhibition. The IC₅₀ values for these excipients, and their mode of inhibitory action for the function of ABCG2 were evaluated and determined.

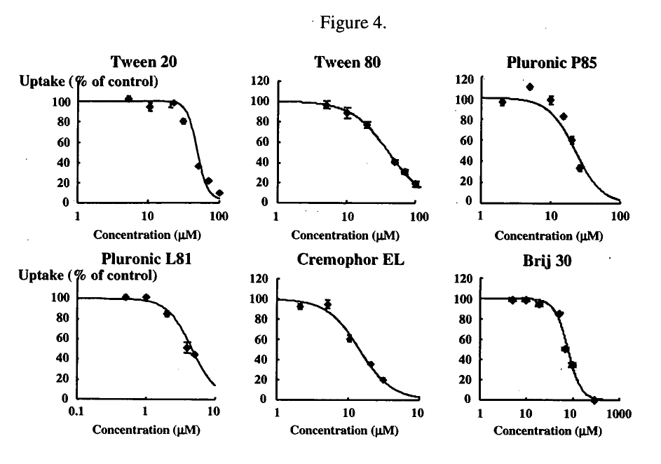

FIG. 4 shows the dose response effects of polyoxyl 35 castor oil, polyoxyethylenesorbitan monolaurate, polyoxyethylenesorbitan monooleate, ethylene oxide/propylene oxide block copolymer; (PEO)₂₆(PPO)_{39.5}(PEO)₂₆, ethylene oxide/propylene oxide block copolymer; (PEO)₂(PPO)40(PEO)₂, and polyoxyl 4 lauryl ether on the uptake of E1S into the vesicles. Results are expressed as means±SE (n=3). These sigmoid curves did not have good fits at the Hill coefficient n=1. Thus, we determined the Hill coefficients, which are shown along with IC₅₀ values in Table 4.

Table 4 shows, inter alia, that the IC₅₀'s of polyoxyl 35 castor oil, polyoxyethylenesorbitan monolaurate and polyoxyl 4 lauryl ether for ABCG2-mediated E1S uptake were 14.4±1.9, 47.6±2.0 and 77.5±4.1 μM, respectively. The Hill coefficients for these were 2.0±0.6, 5.8±1.3 and 3.1±0.6, respectively, suggesting a positive cooperativity in the inhibition of ABCG2 function by these excipients. Such cooperativity is consistent with the solution behavior of the excipients.

TABLE 4

| Excipient | IC₅₀ (μM) | Hill coefficient |
|---|---|---|
| Polyoxyl 35 castor oil | 14.4 ± 1.9 | 2.0 ± 0.6 |
| Polyoxyethylenesorbitan | 47.6 ± 2.0 | 5.8 ± 1.3 |
| monolaurate | | |
| Polyoxyl 4 lauryl ether | 77.5 ± 4.1 | 3.1 ± 0.6 |
| Polyoxyethylenesorbitan | 41.1 ± 1.0 | 1.6 ± 0.1 |
| monooleate | | |
| Ethylene Oxide/Propylene | 22.3 ± 2.2 | 2.8 ± 0.3 |
| Oxide Block Copolymer; | | |
| (PEO)₂₆(PPO)_{39.5}(PEO)₂₆ | | |
| Ethylene Oxide/Propylene | 4.6 ± 0.2 | 2.4 ± 0.2 |
| Oxide Block Copolymer; | | |
| (PEO)₂(PPO)₄₀(PEO)₂ | | |

### Example 5

### Mode of Inhibitory Action of ABCG2 Function by Selected Excipients

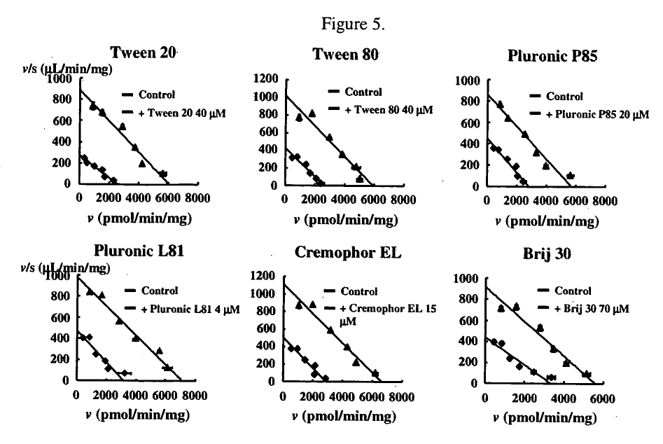

We evaluated the manner of inhibition by the excipients of Example 4 from the results obtained in Example 4. Kₘ and Vₘₐₓ were calculated for ABCG2 inhibition by excipients at a concentration near the IC₅₀ values. Then, their values were compared to those obtained in the absence of excipient. The results are shown in FIG. 5 and Table 5. In Table 5, the values in parentheses are the non-excipients controls. Results are the means±SE (n=3). Vₘₐₓ decreased with use of each excipient, but there was little change in the value of Kₘ. This implies that inhibition manner of polyoxyl 35 castor oil and the other tested excipients is of the non-competitive type.

TABLE 5

| Excipient | Kₘ (μM) | Vₘₐₓ (nmol/min/mg) |
|---|---|---|
| Polyoxyethylenesorbitan | 9.5 ± 1.0 (6.8 ± 0.8) | 2.6 ± 0.2 (6.0 ± 0.4) |
| monolaurate | | |
| Polyoxyethylenesorbitan | 6.2 ± 0.7 (5.8 ± 0.7) | 2.6 ± 0.2 (5.8 ± 0.7) |
| monooleate | | |
| Ethylene Oxide/Propylene | 6.2 ± 0.7 (6.6 ± 0.8) | 2.8 ± 0.2 (5.7 ± 0.4) |
| Oxide Block Copolymer; | | |
| (PEO)₂₆(PPO)_{39.5}(PEO)₂₆ | | |
| Ethylene Oxide/Propylene | 6.6 ± 1.3 (7.3 ± 0.6) | 3.1 ± 0.3 (7.1 ± 0.3) |
| Oxide Block Copolymer; | | |
| (PEO)₂(PPO)₄₀(PEO)₂ | | |
| Polyoxyl 35 castor oil | 5.7 ± 0.9 (6.0 ± 1.0) | 2.9 ± 0.3 (6.6 ± 0.3) |
| Polyoxyl 4 lauryl ether | 7.6 ± 1.4 (6.1 ± 0.8) | 3.4 ± 0.4 (5.6 ± 0.4) |

### Example 6

### Mitoxantrone Accumulation in Cells in vitro

The inhibitory effect of excipients on ABCG2 was further characterized in an intracellular accumulation study. MDCK-II cells overexpressing ABCG2 (BCRP MDCK-II) and, as controls, MDCK-II cells overexpressing green fluorescence protein (GFP) (GFP MDCK-II) were prepared. Mitoxantrone was used as the substrate. Intracellular mitoxantrone accumulation in the presence or absence of excipients was determined. After 2 hr BCRP MDCK-II cells had greatly reduced ritoxantrone accumulation compared with GFP MDCK-II, when measured in the presence of PSC833, a P-gp inhibitor. The reduced accumulation was markedly reversed by GF120918 treatment.

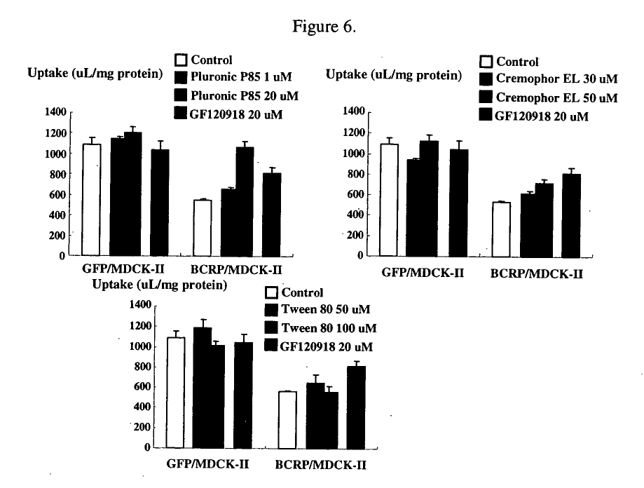

FIG. 6 shows the effect of ethylene oxide/propylene oxide block copolymer, (PEO)₂₆(PPO)_{39.5}(PEO)₂₆; polyoxyethyleneglyceroltriricinoleate 35; and polyoxyethylenesorbitan monooleate, on intracellular mitoxantrone accumulation. All of these excipients significantly inhibited ABCG2 in the vesicle study. Ethylene oxide/propylene oxide block copolymer, (PEO)₂₆(PPO)_{39.5}(PEO)₂₆ and polyoxyethyleneglyceroltriricinoleate 35 increased the mitoxantrone accumulation in BCRP MDCK-II, suggesting that these excipients could inhibit ABCG2 function. On the other hand, there was no significant difference in the mitoxantrone accumulation in BCRP MDCK-II after polyoxyethylenesorbitan monooleate treatment, which did not inhibit ABCG2 in the BCRP MDCK-II. Thus some excipients can have differential effects on ABCG2 inhibition in vesicle studies and cell studies.

### Example 7

### Intracellular Accumulation

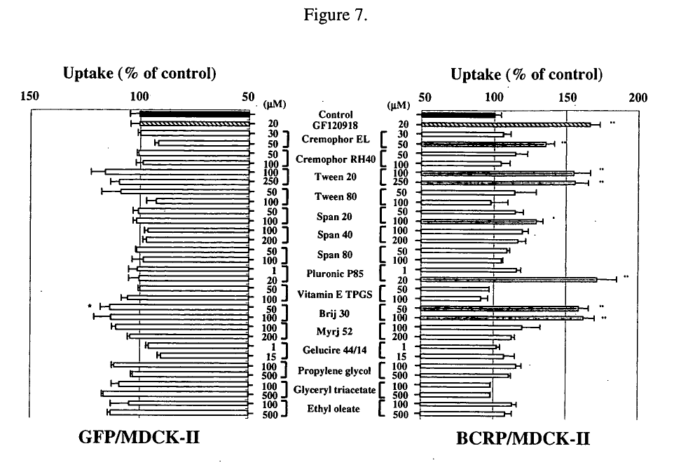

To discover the excipients that can inhibit ABCG2, we performed an intracellular accumulation study using BCRP MDCK-II and GFP MDCK-II cells in the absence or presence of excipients. Mitoxantrone was used as the substrate. To abolish the effect of endogenous P-gp, this experiment was performed in the presence of PSC833. FIG. 7 shows the effect of 15 excipients on the mitoxantrone accumulation in BCRP MDCKII and GFP MDCK-II cells. All of the excipients were used below their cmc, because above their cmc, excipients form micelles that can interact with the substrate, and as a consequence the effective concentration of mitoxantrone in the experimental medium can decrease. The excipients that do not form micelles, such as propylene glycol, glyceryl triacetate and ethyl oleate, were used at concentrations below 500 ,uM. Five excipients: polyoxyethyleneglyceroltriricinoleate 35, polyoxyethylenesorbitan monolaurate, sorbitan monolaurate, ethylene oxide/propylene oxide block copolymer; (PEO)₂₆(PPO)_{39.5}(PEO)₂₆, and lauryl polyethylene glycol ether, significantly increased mitoxantrone accumulation in BCRP MDCK-II cells. Polyoxyethylenesorbitan monolaurate, ethylene oxide/propylene oxide block copolymer; (PEO)₂₆(PPO)_{39.5}(PEO)₂₆, and lauryl polyethylene glycol ether were particularly effective and increased the accumulation in a similar manner to GF120918. These results suggest that the five excipients-can inhibit ABCG2.

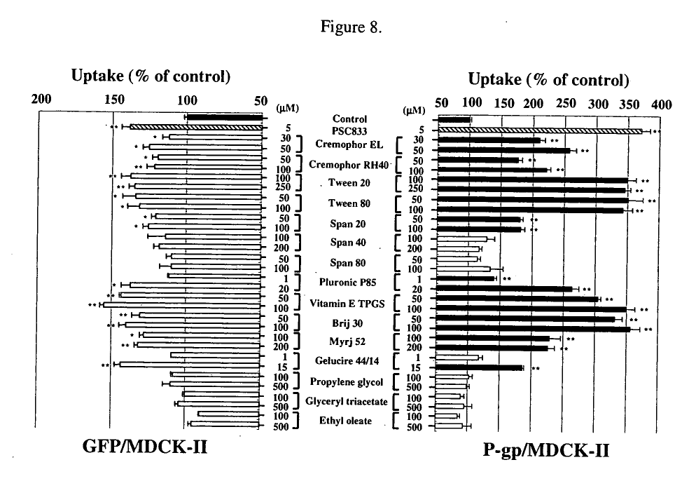

Moreover, to investigate the inhibitory effect of excipients on P-gp, we also performed an intracellular accumulation study for 15 excipients using P-gp MDCK-II cells. Mitoxantrone was again used as the substrate. P-gp MDCK-II cells demonstrated greatly reduced mitoxantrone accumulation compared with GFP MDCK-II cells, which was markedly reversed by PSC833 treatment. PSC833 also reversed mitoxantrone accumulation in GFP MDCK-II cells due to inhibition of endogenous P-gp function in MDCK-II. FIG. 8 shows the effect of 15 excipients on mitoxantrone accumulation in P-gp and GFP MDCK-II. Of the 15 excipients, ten excipients, namely polyoxyethyleneglyceroltriricinoleate 35, polyoxyethyleneglyceroltrihydroxystearate, polyoxyethylenesorbitan monolaurate, polyoxyethylenesorbitan monooleate, sorbitan monolaurate, ethylene oxide/propylene oxide block copolymer; (PEO)₂₆(PPO)_{39.5}(PEO)₂₆, Vitamin E TPGS, lauryl polyethylene glycol ether, polyoxyethylene 40 stearate, and PEG-32 glyceryl laurate, increased mitoxantrone accumulation in P-gp MDCK-II cells, suggesting that these ten excipients can inhibit P-gp function. These excipients also increased mitoxantrone accumulation in GFP MDCK-II cells due to inhibition of endogenous P-gp. These data suggest that some excipients can inhibit both ABCG2 and P-gp and some excipients inhibit only P-gp.

Without being held to any particular mechanism, the different effects of excipients on ABCG2 and P-gp may result from differences in the efflux mechanisms of the two transporters. It has been reported that the efflux of substrate by P-gp occurs from the lipid bilayer. Shapiro A B, Ling V, (1995) Reconstitution of drug transport by purified P-glycoprotein. J Biol Chem: 270, 16167; Shapiro A B, Corder A B, Ling V, (1997) P-glycoprotein-mediated Hoechst 33342 transport out of the lipid bilayer. Eur J Biochem: 250, 115. Efflux by ABCG2 may have another mechanism. In particular, the hypothesis that ABCG2-mediated efflux occurs from the cytoplasm is supported by two experiments. First, several excipients, in particular polyoxyethylene 40 stearate, Vitamin E TPGS, polyoxyethylenesorbitan monooleate, and polyoxyethyleneglyceroltrihydroxystearate inhibited ABCG2 in the vesicle assay, whereas these excipients did not inhibit ABCG2 in the intact cell assay. Thus these excipients can inhibit both ABCG2 and P-gp, at sufficiently high levels of excipient. We propose that excipient concentration levels sufficient to reach the lipid bilayer, i.e., to inhibit P-gp, may be achieved, but at the same time the levels in the cytoplasm remain low, insufficient to inhibit ABCG2. Second, in the time course of initial accumulation of mitoxantrone in P-gp and BCRP MDCK-II cells, PSC833 significantly increased the mitoxantrone accumulation in P-gp MDCK-II cells for the initial 5 min. PSC833 also slightly increased the accumulation in GFP MDCK-II cells probably resulting from inhibition of endogenous P-gp. In contrast, there was no significant difference between BCRP and GFP MDCK-II cells on the initial accumulation of mitoxantrone by GF120918 treatment. PSC833 and GF120918 markedly increased mitoxantrone accumulation for I hr, inhibiting both P-gp and BCRP MDCK-II, respectively. These data suggest that P-gp has faster kinetics than than ABCG2. As an alternative, the mechanism of ABCG2-mediated mitoxantrone efflux may be different from that of P-gp-mediated efflux.

### Example 8

### Effect of Excipients of ATP Levels

The effect of several excipients, in particular ethylene oxide/propylene oxide block copolymer, (PEO)₂₆(PPO)_{39.5}(PEO)₂₆; polyoxyethylenesorbitan monolaurate; polyoxyethyleneglyceroltriricinoleate 35; sorbitan monolaurate; and lauryl polyethylene glycol ether, which inhibit ABCG2, on intracellular ATP levels was measured in BCRP, P-gp, and GFP MDCK-II cells using a luciferin/luciferase assay. These data are shown in Table 6 which shows mean−5.0 (n=3). Sodium azide, used as a positive control, markedly reduced the intracellular ATP in all the cell lines. On the other hand, there was no significant effect by any of the five excipients on the intracellular ATP levels in any of the cell lines.

TABLE 6

| | | GFP/MDCK II | BCRP/MDCK II | P-gp/MDCK II |
|---|---|---|---|---|
| | | (nmol/mg | (nmol/mg | (nmol/mg |
| | | protein) | protein) | protein) |
| Control | | 147.7 ± 7.2 | 133.8 ± 8.5 | 124.0 ± 7.5 |
| Sodium azide | 100 mM | 38.2 ± 1.2 | 24.5 ± 11.7 | 45.6 ± 5.7 |
| Ethylene | 20 μM | 152.8 ± 8.3 | 115.4 ± 10.4 | 112.8 ± 8.9 |
| Oxide/Propylene Oxide | | | | |
| Block Copolymer; | | | | |
| (PEO)₂₆(PPO)_{39.5}(PEO)₂₆ | | | | |
| Polyoxyethylenesorbitan | 250 μM | 150.4 ± 5.2 | 143.4 ± 7.8 | 123.0 ± 5.8 |
| monolaurate | | | | |
| Polyoxyethyleneglycerol- | 50 μM | 154.5 ± 14.2 | 133.6 ± 2.4 | 117.9 ± 9.0 |
| triricinoleate 35 | | | | |
| Sorbitan monolaurate | 100 μM | 132.8 ± 3.6 | 141.9 ± 9.1 | 131.1 ± 7.2 |
| Lauryl polyethylene | 100 μM | 127.5 ± 7.7 | 122.9 ± 10.2 | 123.7 ± 3.5 |
| glycol ether | | | | |

### Example 9

### Plasma Levels, AUC, and Clearance Upon in vivo Administration

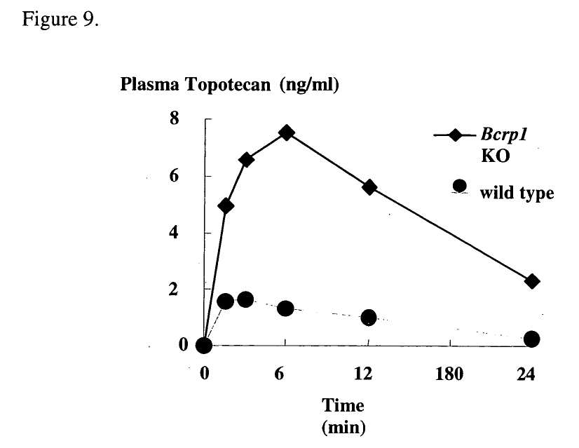

Topotecan (1 mg/kg) was administered orally to wild-type and female bcrp1 KO mice. We then determined the plasma concentration of topotecan as a function of time (FIG. 9). The results are the mean of the measurements The bioavailability of topotecan given orally, as measured by the area under the plasma concentration-time curve (AUC), was more than fivefold higher in bcrp1 KO mice than that in wild-type mice, suggesting that topotecan is a good ABCG2 substrate.

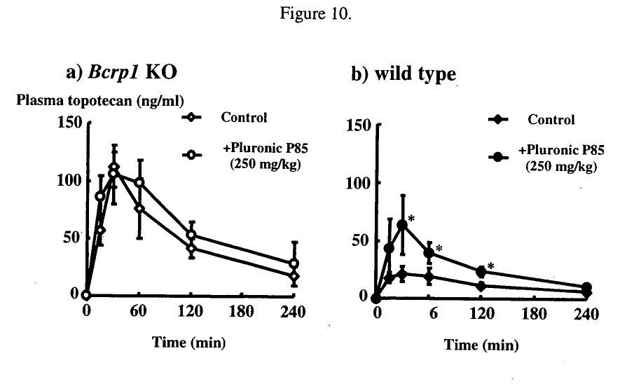

From the drug accumulation study, we found that three excipients strongly inhibited ABCG2, namely ethylene oxide/propylene oxide block copolymer, (PEO)₂₆(PPO)_{39.5}(PEO)₂₆; polyoxyethylenesorbitan monolaurate; and lauryl polyethylene glycol ether. In the present study, we selected ethylene oxide/propylene oxide block copolymer, (PEO)₂₆(PPO)_{39.5}(PEO)₂₆ as test excipient We administered the test excipient or vehicle (phosphate-buffered saline) orally to wild-type and bcrpJ KO mice 15 min before oral administration of topotecan (1 mg/kg). We then determined the plasma concentration of topotecan as a function of time. This result is shown in FIG. 10 and the AUC of plasma topotecan is shown in Table 7 showing means±SD for n=3. Significant changes (p<0.05) in comparison to controls are marked with an asterisk and n.s. means no significant difference. In wild-type mice, the tested excipient significantly increased the plasma concentration of topotecan, whereas it did not affect the bcrp1 KO mice (FIG. 10 and Table 7). Thus, in wild-type mice, the test excipient increased the AUC by about twofold compared with that of control (Table 7), suggesting that ethylene oxide/propylene oxide block copolymer, (PEO)₂₆(PPO)_{39.5}(PEO)₂₆ improves topotecan oral absorption by inhibition of ABCG2.

TABLE 7

| | | AUC | | |
|---|---|---|---|---|
| | | (h | | |
| | | μg/l) | SD | p |
| bcrp1 KO | Control | 195.0 | 30.9 | |
| | +Ethylene Oxide/Propylene Oxide | 243.8 | 64.7 | n.s. |
| | Block Copolymer; | | | |
| | (PEO)₂₆(PPO)_{39.5}(PEO)₂₆ (250 mg/kg) | | | |
| Wild type | Control | 51.7 | 11.7 | |
| | +Ethylene Oxide/Propylene Oxide | 111.6 | 26.8 | 0.05 |
| | Block Copolymer; | | | |
| | (PEO)₂₆(PPO)_{39.5}(PEO)₂₆ (250 mg/kg) | | | |

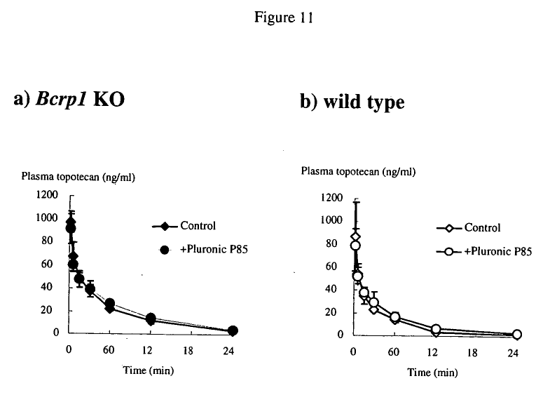

Topotecan was also administered intravenously, for comparison. Ethylene oxide/propylene oxide block copolymer, (PEO)₂₆(PPO)_{39.5}(PEO)₂₆, was given orally to wild-type and bcrp1 KO mice 15 min before intravenous administration of topotecan (1 mg/kg). We then determined the plasma concentration of topotecan as a function of time. These results are shown in FIG. 11 and the AUC of plasma topotecan is shown in Table 8. The excipient had no significant difference upon the plasma levels of topotecan under these conditions. These results suggest that ethylene oxide/propylene oxide block copolymer; (PEO)₂₆(PPO)_{39.5}(PEO)₂₆ given orally does not affect the systemic disposition of topotecan after its intravenous administration.

TABLE 8

| | AUC (h μg/L) | | P |
|---|---|---|---|
| wild type | Control | 415.7 ± 36.2 | |
| | +Ethylene Oxide/Propylene Oxide | 516.8 ± 95.2 | n.s |
| | Block Copolymer; | | |
| | (PEO)₂₆(PPO)_{39.5}(PEO)₂₆ | | |
| Bcrp1 KO | Control | 728.5 ± 30.3 | |
| | +Ethylene Oxide/Propylene Oxide | 804.6 ± 78.5 | n.s. |
| | Block Copolymer; | | |
| | (PEO)₂₆(PPO)_{39.5}(PEO)₂₆ | | |

Clearance values are given in Table 9. The dose of topotecan was 1000 μg/kg of body weight. The value CL_{tot,blood}=CL_{tot,plasma}/R_{B topotecan}, where R_{B} is the ratio of topotecan concentration in blood to plasma, measured as 1.2 in mice.

TABLE 9

| | | CLₜₒₜ,ₚₗₐₛₘₐ | |
|---|---|---|---|
| p.o. | | (L/h/kg) | SD |
| wild type | control | 20.0 | 4.2 |
| | +Ethylene Oxide/Propylene | 9.3 | 2.0 |
| | Oxide Block Copolymer; | | |
| | (PEO)₂₆(PPO)_{39.5}(PEO)₂₆ | | |
| Bcrp1 KO | control | 5.2 | 0.9 |
| | +Ethylene Oxide/Propylene | 4.3 | 1.1 |
| | Oxide Block Copolymer; | | |
| | (PEO)₂₆(PPO)_{39.5}(PEO)₂₆ | | |

| | | CLₜₒₜ,ₚₗₐₛₘₐ | | CLₜₒₜ,_{blood} |
|---|---|---|---|---|
| i.v. | | (L/h/kg) | SD | (L/h/kg) |
| wild type | control | 2.4 | 0.2 | 2.0 |
| | +Ethylene Oxide/Propylene | 1.9 | 0.3 | 1.6 |
| | Oxide Block Copolymer; | | | |
| | (PEO)₂₆(PPO)_{39.5}(PEO)₂₆ | | | |
| BCRP1 KO | control | 1.4 | 0.1 | 1.2 |
| | +Ethylene Oxide/Propylene | 1.2 | 0.1 | 1.0 |
| | Oxide Block Copolymer; | | | |
| | (PEO)₂₆(PPO)_{39.5}(PEO)₂₆ | | | |

Moreover, to evaluate intestinal ABCG2 inhibition by treatment with ethylene oxide/propylene oxide block copolymer, (PEO)₂₆(PPO)_{39.5}(PEO)₂₆, we calculated the quantity Fa*Fg, in which Fa is intestinal absorption and Fg is intestinal metabolism. The product of Fa and Fg is presystemic bioavailability for drugs not further metabolized in the liver. In Table 10, we show clearance values calculated from the data in Tables 7 and 8. We determined Fa*Fg using Eh=CL_{TOT,BLOOD}/Qh where Qh is 5.4 (L/h/kg). The fold change relative to wild type control is also presented. These data indicate that ethylene oxide/propylene oxide block copolymer, (PEO)₂₆(PPO)_{39.5}(PEO)₂₆, given orally, markedly increased Fa * Fg in wild-type mice. On the other hand, Fa * Fg in bcrp1 KO mice was not affected. These results support the inhibition of intestinal ABCG2 by orally administered ethylene oxide/propylene oxide block copolymer, (PEO)₂₆(PPO)_{39.5}(PEO)₂₆.

TABLE 10

| | | | | | Fold |
|---|---|---|---|---|---|
| | F | Eh | Fh | Fa × Fg | Change |
| Wild type | 0.12 | 0.37 | 0.63 | 0.19 | (1) |
| BCRP1 KO | 0.27 | 0.22 | 0.78 | 0.35 | 1.8 |
| wild type + | 0.20 | 0.30 | 0.70 | 0.29 | 1.5 |
| Ethylene Oxide/Propylene | | | | | |
| Oxide Block Copolymer; | | | | | |
| (PEO)₂₆(PPO)_{39.5}(PEO)₂₆ (p.o.) | | | | | |
| Bcrp1 KO + | 0.28 | 0.19 | 0.82 | 0.34 | |
| Ethylene Oxide/Propylene | | | | | |
| Oxide Block Copolymer; | | | | | |
| (PEO)₂₆(PPO)_{39.5}(PEO)₂₆ (p.o.) | | | | | |

### Example 10

### Method of Preparation of Matrix Capsules With Irinotecan

For each preparation a proper quantity of the selected excipient, e.g., polyoxyl 35 castor oil, is melted at 60° C. under magnetic stirring. The required amount of melted excipient (30 mg) is withdrawn by means of a manual pipette (e.g. Brand-Transferpettor or the like) and added to the required quantity of irinotecan (500 mg). The drug is dispersed in the molten matrix under magnetic stirring at 60° C. for two hours. Optionally, polyethylene glycol or the like can be added to aid in dispersion. The dispersion obtained by the above process is then filled into size 00 hard gelatin capsules using a manual pipette.

### Example 11

### Preparation of Capsules Having Prazosin and an ABCG2 Inhibitor

Capsules containing prazosin and an inhibitor of ABCG2 are prepared as follows, based on knowledge of the critical micelle concentration of the inhibitors. Prazosin is purchased from LC Laboratories (Woburn, Mass.) and [³H]-prazosin with an activity of 3000 GBq/mmol is purchased from Perkin-Elmer Life and Analytical Sciences. Approval of use of human subjects is sought from the clinical trial committee.

Prazosin (18 g, 6 μCi) is combined with polyoxyl-4 lauryl ether (2.16 g) and divided into bovine gelatin capsules, pullulan capsules, and hydroxypropyl methyl cellulose capsules at 0.6 g prazosin per capsule. The same amount and activity of prazosin is separately combined with 1.02 g polyoxyl 4 lauryl ether, with 0.46 g polyoxyl 4 lauryl ether, and with 0.27 g polyoxyl 4 lauryl ether. Each formulation is divided into bovine gelatin capsules, pullulan capsules, and hydroxypropyl methyl cellulose capsules at 0.6 g prazosin per capsule.

Prazosin (18 g, 6 μCi) is combined with polyoxyl 35 castor oil (2.94 g) and divided into bovine gelatin capsules, pullulan capsules, and hydroxypropyl methyl cellulose capsules at 0.6 g prazosin per capsule. The same amount and activity of prazosin is separately combined with 1.47 g polyoxyl 35 castor oil, with 0.75 g polyoxyl 35 castor oil, and with 0.36 g polyoxyl 35 castor oil. Each formulation is divided into bovine gelatin capsules, pullulan capsules, and hydroxypropyl methyl cellulose capsules at 0.6 g prazosin per capsule.

Prazosin (18 g, 6 μCi) is combined with polyoxyethylenesorbitan monolaurate (1.81 g) and divided into bovine gelatin capsules, pullulan capsules, and hydroxypropyl methyl cellulose capsules at 0.6 g prazosin per capsule. The same amount and activity of prazosin is separately combined with 0.9 g polyoxyethylenesorbitan monolaurate, with 0.45 g polyoxyethylenesorbitan monolaurate, and with 0.22 g polyoxyethylenesorbitan monolaurate. Each formulation is divided into bovine gelatin capsules, pullulan capsules, and hydroxypropyl methyl cellulose capsules at 0.6 g prazosin per capsule.

The contents of a representative capsule of each type and formulation are suspended in 50, 100, 200, 300, and 400 ml, respectively, of fasted state simulated intestinal fluid for measurement of the critical micelle concentration of the ABCG2 inhibitor by the surface tension method. After the measurement of the critical micelle concentration, a 10 ml aliquot of each sample is centrifuged at 10,000×g for ten minutes and the ratio of soluble and insoluble labeled prazosin is measured. Another three representative capsules of each type are administered to adult volunteers as one oral bolus dose per volunteer for measurement of the time course of prazosin levels in human blood serum. Thereby, the correlation of serum levels of prazosin to the in vitro measurements of the critical micelle concentration is determined.

### Example 12

### Other Useful Excipients

Other useful excipients of the invention will be evident to one of ordinary skill in the art, including, but not limited to those listed in Table 11.

TABLE 11

| FlaskN° | Excipients Name | Commercial Name |
|---|---|---|
| 1 | Ethyl oleate | Kessco EO |
| 2 | Vitamin E TPGS | N.A. |
| 3 | Polysorbate 80 | Montanox 80 |
| 4 | Polyoxyl 40 hydrogenated castor oil | Cremophor RH40 |
| 5 | Glyceryl triacetate | Triacetin |
| 6 | Glyceryl monolinoleate | Maisine 35-1 |
| 7 | Lauryol PEG-32 glycerides | Gelucire 44/14 |
| 8 | Glycerol Monostearate | Cithrol GMS 0400 |
| 9 | Polyoxyl 10 oleyl ether | Brij 96V |
| 10 | Sorbitan monopalmitate | Montane 40 |
| 11 | PEG-6 oleoyl glycerides | Labrafil M1944CS |
| 12 | PEG-8 caprylic/capric glycerides | Labrasol |
| 13 | Propylene glycol monolaurate | Lauroglycol 90 |
| 14 | Sorbitan trioleate | Crill 45 R |
| 15 | Sorbitan monooleate | Montane 80VGPha |
| 16 | Sorbitan monolaurate | Montane 20VGPha |
| 17 | PEG 6000 | N.A. |
| 18 | Propylene Glycol | N.A. |
| 19 | Diethylene glycol monoethyl ether | Transcutol HP |
| 20 | Poloxamer 124 | Pluronic L44 |

All cited references are incorporated herein in their entirety for all purposes.

We claim:

1. A method of enhancing absorption of a pharmaceutical agent comprising administering said agent to a subject, in combination with an inhibitor of ABCG2, wherein the amount of the inhibitor is less than or about the critical micelle concentration of the inhibitor when delivered to a mucosal surface of the subject.

2. The method of claim 1 wherein the agent is administered to a gastrointestinal tract of the subject.

3. The method of claim 1 wherein the inhibitor is selected from the group consisting of polyoxyethyleneglyceroltriricinoleate 35; polyoxyethylenesorbitan monolaurate; lauryl polyethylene glycol ether; ethylene oxide/propylene oxide block copolymer (PEO)₂₆(PPO)_{39.5}(PEO)₂₆; and ethylene oxide/propylene oxide block copolymer (PEO)₂(PPO)₄₀(PEO)₂; or combinations thereof.

4. The method of claim 1, wherein the agent is a chemotherapeutic agent.

5. The method of claim 1, further comprising administering said agent in combination with an effective amount of reserpine, CI 1033, GF 120918, fumitremorgin C, Ko 134 or Ko 132.

6. The method of claim 1 which results in at least about 60 % inhibition of ABCG2.

7. An oral dosage composition for mucosal administration comprising a pharmaceutical agent and an excipient capable of inhibiting ABCG2 wherein a concentration of said excipient is less than or about a critical micelle concentration of said excipient when delivered enterically.

8. The oral dosage composition of claim 7 wherein the concentration of said excipient is about one-half of the critical micelle concentration of said excipient.

9. The oral dosage composition of claim 7 wherein the concentration of said excipient is about one-quarter of the critical micelle concentration of said excipient.

10. The oral dosage composition of claim 7 wherein the concentration of said excipient is about one eighth of the critical micelle concentration of said excipient.

11. The oral dosage composition of claim 7 wherein the concentration of said excipient is between about one-twentieth of the critical micelle concentration of said excipient and about one-fifth of the critical micelle concentration of said excipient.

12. The oral dosage composition of claim 7 wherein the concentration of said excipient is between about one-eighth of the critical micelle concentration of said excipient and the critical micelle concentration of said excipient.

13. The oral dosage composition of claim 7 wherein the concentration of said excipient is between about one-eighth of the critical micelle concentration of said excipient and one-half of the critical micelle concentration of said excipient.

14. The oral dosage composition of claim 7 wherein the concentration of said excipient is between about one-eighth of the critical micelle concentration of said excipient and one-quarter the critical micelle concentration of said excipient.

15. The oral dosage composition of claim 7 wherein the concentration of said excipient is between about one-quarter of the critical micelle concentration of said excipient and one-half of the critical micelle concentration of said excipient.

16. The oral dosage composition of claim 7 wherein the concentration of said excipient is between about one-half of the critical micelle concentration of said excipient and the critical micelle concentration of said excipient.

17. The oral dosage composition of claim 7 further comprising a semi-solid matrix comprising a lecithin.

18. The oral dosage composition of claim 7 further comprising a semi-solid matrix comprising a polyglycolized glyceride.

19. The oral dosage composition of claim 18 wherein the semi-solid matrix further comprises a lecithin.

20. A capsule comprising a pharmaceutical agent and an effective amount of an inhibitor of ABCG2 wherein when administered enterically a concentration of said inhibitor is about a critical micelle concentration of said inhibitor.
